(19) United States
(12) Patent Application Publication
(10) Pub. No.: US 2025/0009608 A1
(43) Pub. Date: Jan. 9, 2025

(54) Radically Polymerizable Materials For The Production Of Dental Moldings With High Fracture Toughness

(71) Applicant: Ivoclar Vivadent AG, Schaan (LI)

(72) Inventors: Norbert MOSZNER, Triesen (LI); Yohann CATEL, Sevelen (CH); Pascal FÄSSLER, Mels (CH); Kai RIST, Feldkirch (AT)

(21) Appl. No.: 18/757,346

(22) Filed: Jun. 27, 2024

(30) Foreign Application Priority Data
Jun. 30, 2023 (EP) 23182868.2

(51) Int. Cl. A61K 6/896; A61K 6/887; C08F 222/22

(52) U.S. Cl. CPC A61K 6/896; A61K 6/887; C08F 222/22

(57) ABSTRACT

A radically polymerizable composition including at least one urethane group-containing (meth) acrylate monomer, at least one fracture toughness modifier and at least one transfer reagent of the general formula I

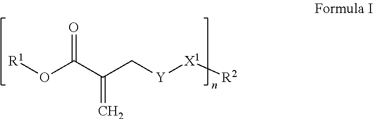

The composition is particularly suitable as dental materials, for example for the production of dental moldings by generative processes, and is characterised by a high fracture toughness.

### CROSS-REFERENCE TO RELATED APPLICATIONS

This application claims priority to European Patent Application No. 23182868.2 filed on Jun. 30, 2023, the disclosure of which is incorporated herein by reference in its entirety.

### TECHNICAL FIELD

The present invention relates to radically polymerizable compositions which are particularly suitable as dental materials for the production of dental moldings, such as artificial teeth, dentures, inlays, onlays, splints, crowns, bridges, veneering materials and orthodontic appliances by generative processes.

### BACKGROUND

Classical dental polymerization systems usually consist of a mixture of monomers, initiator components, stabilizers and pigments (J. Viohl, K. Dermann, D. Quast, S. Venz, Die Chemie zahnärztlicher Füllungskunststoffe [The chemistry of dental restoratives], Carl Hanser Verlag [Carl Hanser Publishers], Munich-Vienna 1986, 21-27). In this context, mixtures of dimethacrylates are mostly used as monomers for the construction of polymer networks (cf. A. Peutzfeldt, Resin composites in dentistry: The monomer systems, Eur. J. Oral. Sci. 105 (1997) 97-116; N. Moszner, T. Hirt, New Polymer-Chemical Developments in Clinical Dental Polymer Materials: Enamel-Dentin Adhesives and Restorative Composites, J. Polym. Sci. Part A: Polym. Chem. 50 (2012) 4369-4402). Examples include the highly viscous dimethacrylates 2,2-bis[4-(2-hydroxy-3-methacryloyloxypropyl)phenyl]propane (Bis-GMA) and 1,6-bis[2-methacryloyloxy ethoxy carbonylamino]-2,4,4-trimethylhexane (UDMA) and the low-viscosity dimethacrylates bismethacryloyloxymethyltricyclo[5.2.1.] decane (TCDMA), decanediol-1,10-dimethacrylate (D₃ MA) and triethylene glycol dimethacrylate (TEGDMA). In contrast, the monofunctional monomer methyl methacrylate (MMA), which has a low viscosity but is very volatile, is mainly used for dental prostheses.

Depending on the field of application, further additives can be added. To initiate radical polymerization by light, photoinitiators for the UVA range or visible range are used, which form radicals when irradiated with UVA light, usually with a wavelength of 365 nm (Hg vapor lamp) to 395 nm (LED), or blue light (400-480 nm).

Prosthetic materials contain powdered polymethyl methacrylate (PMMA) as an essential component, which forms a paste with MMA. Curing takes place thermally or with redox initiators.

A major problem in the radical polymerization of methacrylates is the polymerization shrinkage (ΔV_{P}), i.e. the volume contraction of the methacrylate monomers used during polymerization, which can lead, for example, to very disadvantageous marginal gap formation in filling composites or adversely affects the dimensional stability of denture materials. The polymerization shrinkage ΔV_{P} is e.g. 21.0% by volume for pure MMA.

Another disadvantage of PMMA or dimethacrylate polymers is the high brittleness of the materials. The low fracture toughness is an inherent property of the amorphous PMMA glass and, in the case of polymer networks formed by dimethacrylate mixtures, is mainly caused by their irregular network structure. In addition, methacrylates exhibit incomplete double bond conversion, which in the case of monomethacrylates may result in a significant residual monomer content and thus adversely affect the biocompatibility of the polymers.

Additive manufacturing (AM) processes are increasingly being used to produce dental molded bodies. These are also referred to as generative manufacturing processes or 3D printing and are summarized under the term “rapid prototyping” (RP). Here, three-dimensional models or components are continuously produced layer by layer on the basis of computer generated design data (CAD data). Common processes are stereolithography (SL), selective laser sintering (SLS), 3D printing, fused deposition modelling (FDM), ink jet printing (IJP), 3D plotting, multi-jet modelling (MJM), solid freeform fabrication (SFF), laminated object manufacturing (LOM) and laser powder forming (LPF). These processes can be used to produce models, components or molded parts cost-effectively, even in small series (A. Gebhardt, Generative Fertigungsverfahren [Generative manufacturing processes], 3^{rd} ed., Carl Hanser Verlag [Carl Hanser Publishers], Munich 2007, 77ff.).

In stereolithography, a molded part is built up layer by layer from a liquid and curable monomer resin on the basis of CAD data, whereby the curing of the layers takes place through controlled light exposure and thus through photopolymerization (cf. I. Gibson, D. W. Rosen et al. Gibson, D. W. Rosen, B. Stucker et al, “Additive Manufacturing Technologies”, Volume 238, Springer Verlag [Springer Publishers] (2010)). A UV laser is often used as a light source for this purpose. The DLP (Digital Light Processing) process uses a projectable image to cure the materials layer by layer.

For technical applications, photopolymerization resins based on acrylates or epoxides, which are characterized by high reactivity, are often used. Such monomer systems are not suitable for intraoral curing because of the mutagenic properties of many acrylates and epoxides. Methacrylates are usually used here, which have a significantly better biocompatibility but a much lower photopolymerization reactivity compared to acrylates.

In view of the special characteristics of this process, construction materials for the generative production of dental molded parts must meet a number of different requirements. They should have low viscosity and high transparency and have good mechanical properties after curing. The manufactured molded articles must have good flexural strength and a high modulus of elasticity and, in particular, good fracture toughness.

An improvement in fracture toughness can be achieved by internal plasticization, e.g. by adding flexible monomers. These have the disadvantage that they significantly reduce the flexural strength and the modulus of elasticity of the polymers. Furthermore, so-called impact modifiers can be added to polymerization resins. These are usually polymer particles with a core-shell structure.

WO 2014/078537 A1 and corresponding US 2014131908 A1, which US published application is hereby incorporated by reference, disclose resin blends for the production of dental moldings by 3D printing processes based on mono- and multifunctional methacrylates, which obtain silicone acrylate-based impact modifiers with core-shell structure to improve impact strength and fracture toughness.

US 2018/0000570 A1, which is hereby incorporated by reference, relates to construction materials based on mono- and multifunctional (meth)acrylates for the generative production of dental components. The construction materials contain silicone-acrylate-based rubber particles with a core-shell structure as impact modifiers and oligomers, which are produced by reacting trimethyl-1,6-diisocyanate, bisphenol-A-propoxylate and 2-hydroxyethyl methacrylate (HEMA). The cured components are said to have good mechanical and physical properties as well as good biocompatibility.

U.S. Pat. No. 10,299,896 B2 and US 2019/0053883 A1, which are both hereby incorporated by reference, disclose dental components produced by generative processes comprising at least two layers of differently composed construction materials. One layer is formed by a material comprising oligomers obtained by reacting intermediates having terminal isocyanate groups with hydroxyl-based methacrylates, a polymerizable acrylic compound and an impact modifier. At least one further layer is formed by a material containing a urethane monomer, a glycol dimethacrylate and filler. The combination of materials with different mechanical and physical properties is said to be advantageous for adapting the components to different requirements. Commercially available polymers with a core-shell structure are used as impact modifiers.

EP 3 564 282 A1 and corresponding U.S. Ser. No. 11/542,362B2, which US patent is hereby incorporated by reference, disclose curable compositions for high temperature photopolymerization processes containing an oligomeric urethane dimethacrylate as a glass transition temperature modifier, a (poly)carbonate (poly)urethane dimethacrylate as a toughness modifier and optionally core-shell particles. They are said to have good thermomechanical properties and biocompatibility and be suitable for the fabrication of orthodontic appliances.

The mode of action of impact modifiers with a core-shell structure is based on an interaction of the tip of a forming crack with the polymer particles. The particles have a relatively soft polymer core and a hard polymer shell. When a crack tip hits such a particle, a cavity is formed in the core and the polymer shell separates from the polymerized resin matrix and the core from the shell, resulting in the formation of space (cavitation) for plastic deformation surfaces. An improvement in impact strength is not equivalent to an improvement in fracture toughness. Core-shell particles impart relatively good flexural strength and modulus of elasticity to materials, but are difficult to disperse in monomer mixtures. Another major disadvantage for dental applications is that core-shell polymers (CSPs) significantly reduce the transparency of the materials, which has a detrimental effect on the stereolithographic construction process. A reduction in transparency also reduces the depth of cure and can thus lead to a deterioration of the mechanical properties and an increase in the residual monomer content.

From EP 4 085 893 A1 and corresponding U.S. Pat. No. 11,998,621B2, which US patent is hereby incorporated by reference, radically polymerizable materials for the production of dental moldings by generative processes are known, which contain an ABA or AB block copolymer to improve the fracture toughness and fracture work. The materials preferably also contain 30 to 70 wt. % of at least one monofunctional, free-radically polymerizable monomer and at least one free-radically polymerizable urethane di(meth)acrylate-telechel having a number average molecular weight of 750 to 2000 g/mol. The materials may contain core-shell polymers to improve fracture toughness and impact strength. They are characterized by a relatively high content of monofunctional monomers.

### SUMMARY

According to the invention, it was found that a significant improvement in fracture toughness by using block copolymers or core-shell polymers can only be achieved with polymer networks with a lower crosslink density. A low cross-linking density requires the addition of considerable amounts of monofunctional monomers, which can lead to an increased residual monomer content and thus to toxicological problems. In addition, the use of monofunctional monomers is associated with a deterioration of the Young's modulus.

It is an object of the invention to provide materials for the production of dental moldings with good fracture toughness and high fracture work. The materials should have a low viscosity and good biocompatibility and, after curing, should have good mechanical properties, in particular a high flexural strength and a high flexural modulus, after water storage. The materials should also be fast curing and show high monomer conversion.

### DETAILED DESCRIPTION

According to the invention, this object is achieved by radically polymerizable compositions comprising at least one urethane group-containing (meth)acrylate monomer, at least one fracture toughness modifier and at least one transfer reagent of the general formula I:

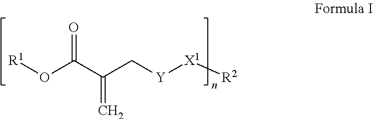

in which the variables have the following meanings:
R¹ is hydrogen, an aliphatic linear or branched C₁-C₁₅ alkyl radical which can be interrupted by one or more, preferably 1 to 3, O or S, and/or can be unsubstituted or substituted by one or more, preferably 1, C₁-C₅ alkyl groups and/or OH, or is benzyl or phenyl which can be unsubstituted or substituted by one or more, preferably one, C₁-C₃ alkyl groups;
Y is S or SO₂;
X¹ is absent or is an aliphatic linear or branched C₁-C₁₅ alkyl radical which can be interrupted by an ester group (—CO—O—), urethane group (—O—CO—NH—), or one or more, preferably 1 to 3, O or S, and/or can be unsubstituted or substituted by one or more, preferably one, C₁-C₅ alkyl groups and/or OH;
R² is an aliphatic linear or branched C₁-C₂₀-alkyl radical which can be interrupted by an ester group (—CO—O—), urethane group (—O—CO—NH—), or one or more, preferably 1 to 3, O or S, and/or can be unsubstituted or substituted by 1 to 3 OH groups, or is a cycloaliphatic C₅-C₂₀ radical, an aromatic C₆-C₂₀ radical or an isocyanuric acid radical, each of which can be unsubstituted or substituted by one or more, preferably one, C₁-C₅ alkyl groups;
n is 1, 2 or 3.

Preferred compounds of formula I are those in which the variables have the following meanings:
R¹ is aliphatic linear or branched C₁-C₁₀-alkyl radical which can be interrupted by 1 to 3 O or S, and/or can be unsubstituted or substituted by 1 to 3 methyl groups;
Y is S or SO₂;
X¹ is absent or is an aliphatic linear or branched C₁-C₁₀-alkyl radical which can be interrupted by an ester group (—CO—O—), urethane group (—O—CO—NH—), or 1 to 3 O or S, and/or can be unsubstituted or substituted by 1 to 3 methyl groups;
R² is an aliphatic linear or branched C₁-C₁₆ alkyl radical which can be interrupted by an ester group (—CO—O—), urethane group (—O—CO—NH—), or 1 to 3 O or S, and/or can be substituted by an OH group, or is an aromatic C₆-C₂₀ radical which is unsubstituted or substituted by a methyl group;
n is 1 or 2.

Particularly preferred are compounds of the formula I in which the variables have the following meanings:
R¹ is an aliphatic linear or branched C₁-C₆ alkyl radical which can be interrupted by one or more, preferably 1 to 2, O or S, and is preferably a linear C₁-C₃ alkyl radical;
Y is S or SO₂, preferably SO₂;
X¹ not applicable;
R² is an aliphatic linear or branched C₁-C₁₂ alkyl radical which can be interrupted by an ester group (—CO—O—), urethane group (—O—CONH—), or 1 to 2 O or S, and/or can be unsubstituted or substituted by an OH group, or is an aromatic C₆-C₂₀ radical, preferably a methyl-substituted phenyl group;
n is 1.

All the formulae shown here extend only to those compounds that are compatible with the chemical valence theory. The indication that a radical is interrupted, e.g. by one or more oxygen atoms, is to be understood in such a way that these atoms are each inserted into the carbon chain of the radical. These atoms are thus bounded on both sides by C atoms and cannot be terminal. C₁ radicals cannot be branched or interrupted. According to the usual nomenclature, aromatic hydrocarbon radicals are also understood to be radicals that contain aromatic and non-aromatic groups. A preferred aromatic radical is, for example, the p-tolyl group -Ph-CH₃.

Transfer reagents of formula I are known in part and can be prepared, for example, in the manner described in WO 2016/005534 A1 and corresponding U.S. Ser. No. 10/342,744B2, which US patent is hereby incorporated by reference, or WO 2016/177677 A1 and corresponding U.S. Ser. No. 10/982,071 B2 which US patent is hereby incorporated by reference, or analogously. Preferred examples of compounds of formula I are:

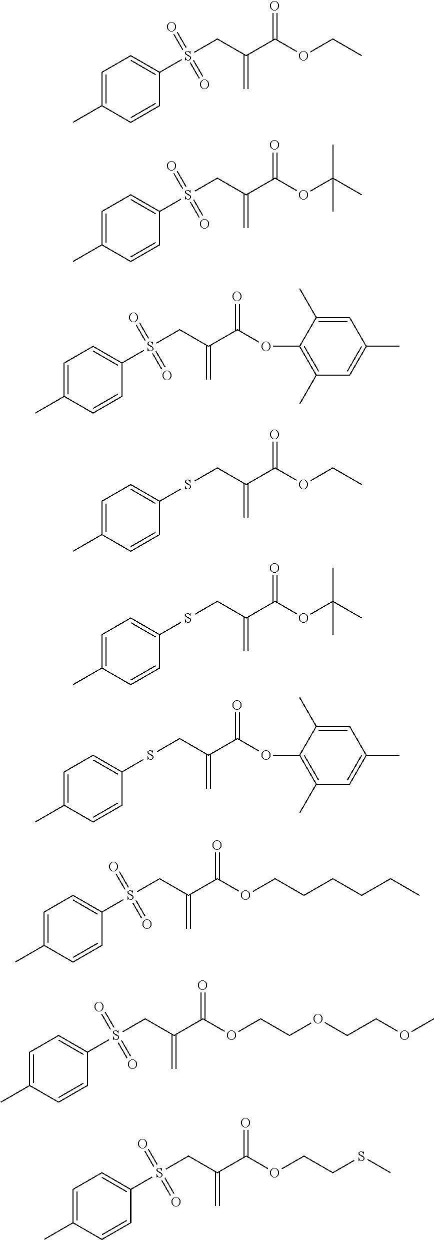

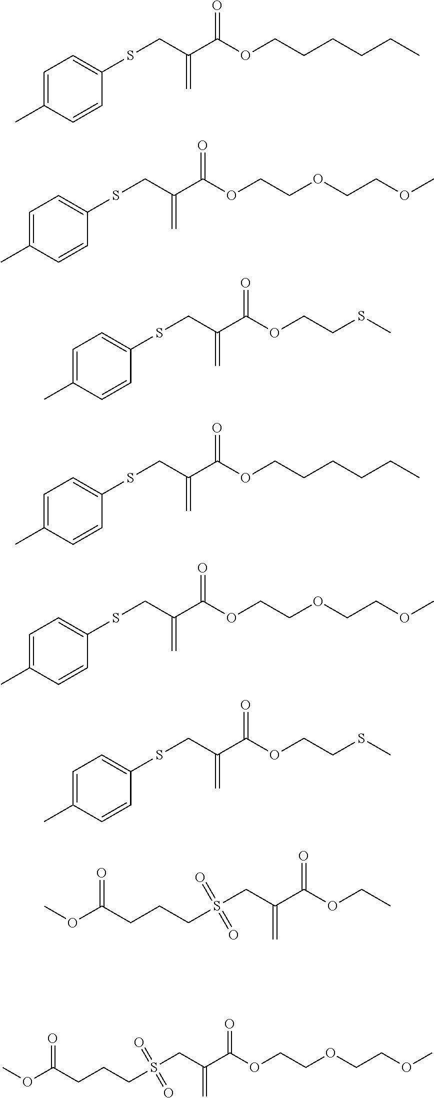

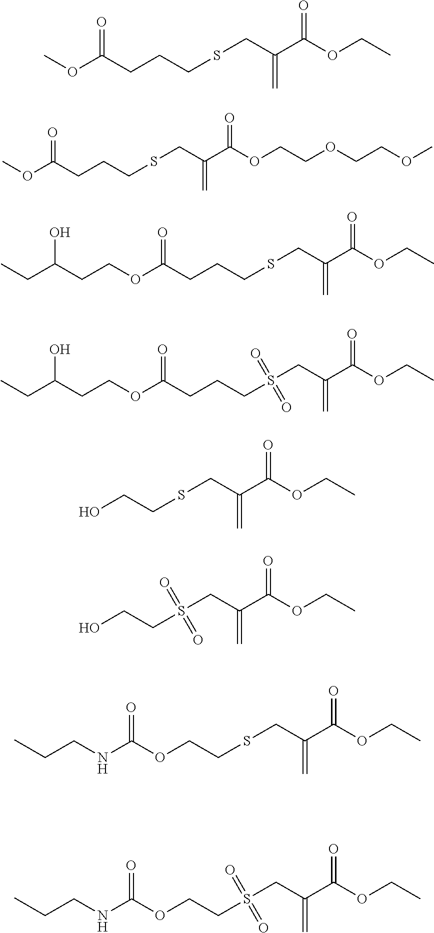

The urethane group-containing (meth)acrylate monomer is preferably a monomer or a mixture of monomers having a urethane group content of 0.01 to 5 mmol/g, preferably 0.1 to 4.5 mmol/g and more preferably 0.5 to 4.0 mmol/g. Particularly preferred are urethane di(meth)acrylates, especially urethane di(meth)acrylates with a molar mass of less than 2000 g/mol, and most preferably urethane di(meth)acrylates with a molar mass of less than 2000 g/mol, which contain 1 to 4 urethane groups.

Urethane group-containing (meth)acrylate monomers preferred according to the invention are obtainable by:
A) Reaction of 2 moles of 2-isocyanatoethyl(meth)acrylate (R⁴: H or CH₃) with one mole of α,ω-alkanediols, cycloaliphatic, tricyclic or aromatic diols as well as ethoxylated or propoxylated aromatic diols (R³: C₁-C₂₀ aliphatic, cycloaliphatic or tricyclic alkylene or C₆-C₁₄-arylene or alkoxylated C₆-C₁₄-arylene):

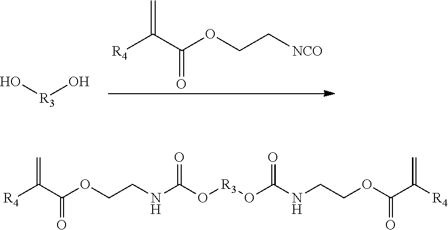

B) Reaction of 1 mole of one or more diisocyanates (R⁵: C₁-C₁₂ aliphatic, cycloaliphatic alkylene or C₆-C₁₄ arylene which may carry alkyl substituents) with 2 moles of 2-hydroxyalkyl(meth)acrylate, poly(ethylene glycol) or poly(propylene glycol) mono(meth)acrylate and mixtures thereof (R⁶: H or CH₃; R⁷: linear or branched C₁-C₄₀ alkylene, wherein the alkylene chain may be interrupted by one or more O atoms):

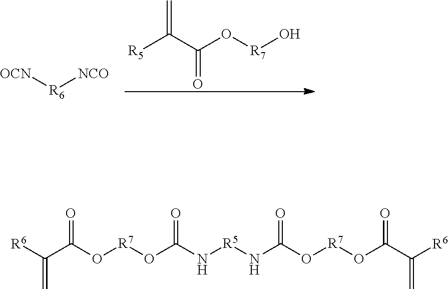

C) Reaction of 1 mol of ethoxylated or propoxylated bisphenol-A, decanediol or dodecanediol or other α,ω-alkanediols, cycloaliphatic, tricyclic or aromatic diols, or ethoxy- or propoxylated aromatic diols (R³: C₁-C₂₀ aliphatic, cycloaliphatic or tricyclic alkylene or C₆-C₁₄-arylene or alkoxylated C₆-C₁₄-arylene) with 2 moles of isophoronediisocyanate (IPDI) and subsequent reaction with 2 moles of 2-hydroxyethyl(meth)acrylate or hydroxypropyl(meth)acrylate (R⁶: H or CH₃; R⁷: linear or branched C₁-C₄₀-alkylene, wherein the alkylene chain may be interrupted by one or more O atoms):

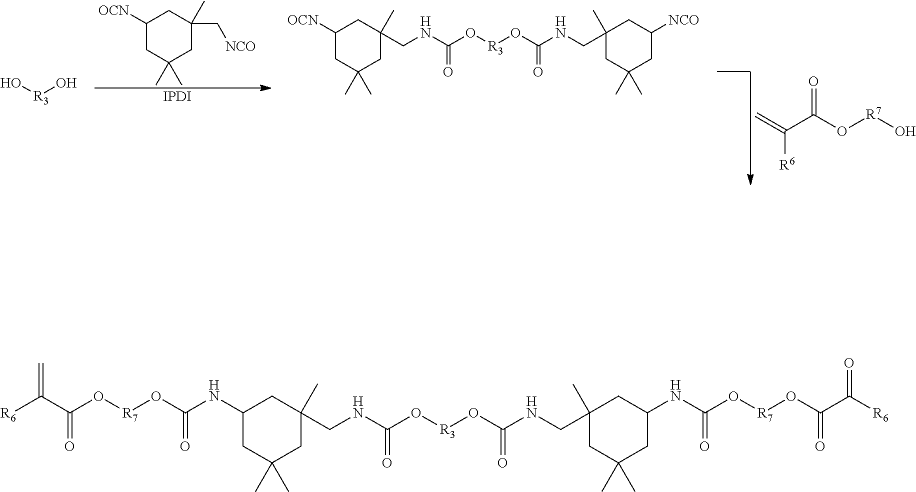

D) Reaction of 1 mol of 2-isocyanatoethyl(meth)acrylate with 1 mol of 2-hydroxyalkyl(meth)acrylate, poly(ethylene glycol) or poly(propylene glycol) mono(meth)acrylate (R⁷: linear or branched C₁-C₄₀ alkylene, wherein the alkylene chain may be interrupted by one or more O atoms; R⁴, R⁶: H or CH₃):

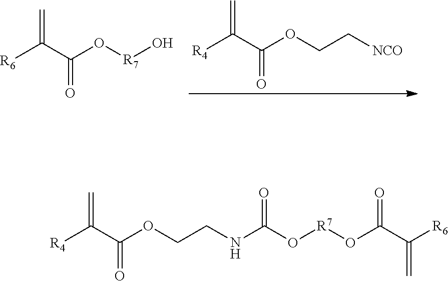

Particularly preferred according to the invention are urethane di(meth)acrylates with a molar mass of less than 750 g/mol, which are obtainable according to synthesis path A by:

Reaction of 2 moles of 2-isocyanatoethyl(meth)acrylate with one mole of α,ω-alkanediols (ethylene glycol, propylene glycol, butane-1,4-diol, pentane-1,5-diol, neopentyl glycol, hexane-1,6-diol, 2,2,4-timethylhexanediol, 2,2,4-trimethyl-1,3-pentanediol, heptane-1,7-diol, octane-1,8-diol, nonane-1,9-diol, decane-1,10-diol, undecane-1,11-diol or dodecane-1,12-diol), C₆-C₁₈-alkanediols, which can contain 1 to 4 O or S atoms in the carbon chain, cycloaliphatic diols (2-methyl-1,4-cyclopentanediol, 1,3- or 1,4-cyclohexanediol, cyclooctanediol, methyl cycloheptanediol, 2,5-dimethyl-1,4-cyclohexanediol, 3,3,5-trimethylcyclo pentane-1,2-diol, 2,2,4,4-tetramethyl-1,3-cyclobutanediol), tricyclic (tricyclodecanedimethanol: tricyclo[5.2.1.0^{2,6}]decanedimethanol) or aromatic, ethoxy- or propoxylated diols with on average 2, 3 or 4 ethoxy or propoxy groups (bisphenol-A, bisphenol-B, bisphenol-E, bisphenol-F, bisphenol-S, hydroquinone, resorcinol, 2,6-dihydroxynaphthalene or 4,4′-dihydroxybiphenyl).

Also particularly preferred are urethane di(meth)acrylates with a molar mass of less than 750 g/mol, which are obtainable according to synthesis path B by:

Reaction of 1 mol of hexamethylene-1,6-diisocyanate (HMDI), 2,2,4-trimethyl hexamethylene-1,6-diisocyanate (TMDI), isophoronediisocyanate (IPDA), diphenylmethane-4,4′-diisocyanate (4,4′-MDI), hydrogenated diphenylmethane-4,4′ diisocyanate (H₁₂-MDI: 4,4′-methylene bis(cyclohexylisocyanate) or tetramethyl xylylenediisocyanate (TMXDI: 1,3-bis(2-isocyanatopropan-2-yl)benzene) or mixtures thereof with 2 moles of 2-hydroxyethyl(meth)acrylate, hydroxypropyl(meth)acrylate, poly(ethylene glycol)-200-mono(meth)acrylate or poly(propylene glycol)-200-mono(meth)acrylate.

Other preferred urethane di(meth)acrylates are the urethane di(meth)acrylate telechels disclosed in EP 4 085 893 A1 with a molecular weight of 800 to 2000 g/mol. These are obtainable by reacting diisocyanates with diols (HO-DA-OH) and subsequently reacting the α,ω-isocyanate-functionalized urethane-telechels with 2-hydroxyethylmethacrylate (HEMA) or hydroxypropylmethacrylate (HPMA). DA stands for an aromatic or aliphatic hydrocarbon radical having 6 to 33 carbon atoms, preferably a divalent polycyclic hydrocarbon radical, in particular an o-diphenyl, p-diphenyl or bisphenol A radical, or a branched or preferably linear C₂-C₁₈ alkylene group. The hydrocarbon radicals may contain one or more O atoms and/or S atoms, with O atoms being preferred.

Preferred diols of the formula HO-DA-OH are ethoxylated or propoxylated bisphenol-A, o-diphenyl or p-diphenyl with 2 to 6 ethoxy or propoxy groups as well as C₂-C₁₈-alkanediols which can contain 1 to 4 O or S atoms in the carbon chain. Particularly preferred diols are ethoxylated or propoxylated bisphenol-A with 2, 3 or 4 ethoxy or propoxy groups, hexane-1,6-diol, octane-1,8-diol, nonane-1,9-diol, decane-1,10-diol or dodecane-1,12-diol, tetra- or pentaethylene glycol. Ethoxylated or propoxylated bisphenol-A with 2 or 3 ethoxy or propoxy groups, decanediol, undecanediol or dodecanediol as well as cyclic or polycyclic aliphatic diols, in particular cyclohexanediol, norbornanediol, tricyclodecanediol and tricyclodecanedimethanol (octahydro-4,7-methano-1H-indendimethanol) are particularly preferred.

Preferred diisocyanates are hexamethylene-1,6-diisocyanate (HMDI), 2,2,4-trimethylhexamethylene-1,6-diisocyanate (TMDI), 1-isocyanato-3-isocyanatomethyl-3,5,5-trimethylcyclohexane (isophorone diisocyanate, IPDI), m-tetramethylxylylene diisocyanate, (1,3-bis(2-isocyanato-2-propyl)benzene, TMXDI), toluene-2,4-diisocyanate (TDI), diphenylmethane-4,4′-diisocyanate (MDI) and 1-isocyanato-4-[(4-isocyanatocyclohexyl)methyl]cyclohexane (H₁₂ MDI), IPDI being particularly preferred.

Particularly preferred are telechels according to the general formula (II)

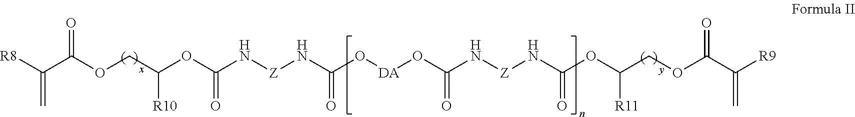

in which the variables have the following meanings:
R⁸, R⁹ independently of each other are each H or methyl, preferably methyl,
R¹⁰, R¹¹ independently of each other are each H or methyl, preferably methyl,
x, y independently of each other, are each an integer from 1 to 11, preferably 1 to 5,
n is 1, 2 or 3, preferably 1,
DA is a structural element derived from the diols HO-DA-OH by splitting off the hydrogen atoms of the two hydroxyl groups,

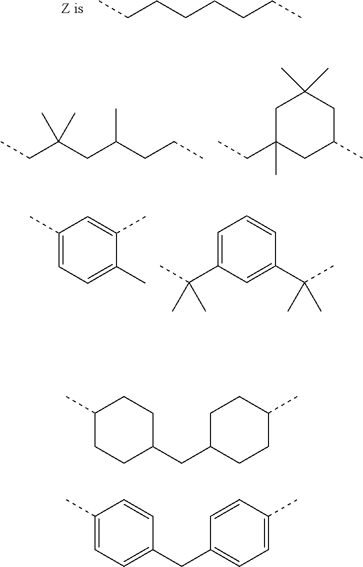

preferably:

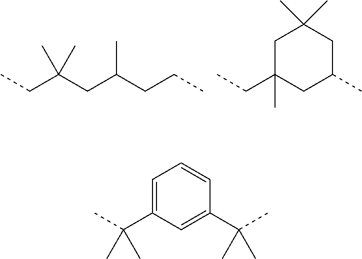

The urethane di(meth)acrylate telechels carry two radically polymerizable (meth)acrylate groups and are suitable as crosslinkers. They are characterized by a good radical polymerization ability and, due to their relatively high molar mass, yield polymers with a lower network density and low polymerization shrinkage.

Very particularly preferred urethane di(meth)acrylates are UDMA (1,6-bis-[2-methacryloyloxyethoxycarbonylamino]-2,4,4-trimethylhexane=addition product of 1 mol 2,2,4-trimethylhexamethylene-1,6-diisocyanate and 2 mol 2-hydroxyethylmeth-acrylate), V-380 (addition product of 1 mol α,α,α′,α′-tetramethyl m-xylylene diisocyanate and a mixture of 1.4 mol 2-hydroxyethylmethacrylate and 0.6 mol 2-hydroxypropylmethacrylate) and V-818 (addition product of 1 mol tricyclo[5.2.1.0^{2,6}]decanedimethanol and 2 mol 2-isocyanatoethylmethacrylate). UDMA has a urethane group content of 4.235 mmol/g.

The compositions according to the invention comprise at least one fracture toughness modifier, preferably at least one block copolymer.

Block copolymers are macromolecules consisting of two or more covalently bonded co- or preferably homopolymer blocks. Block copolymers preferred according to the invention are AB diblock and in particular ABA triblock copolymers.

The A block is a polymerizate, preferably an oligomer, composed of one or more of the following monomers: cyclic aliphatic esters or ethers, arylene oxide, alkylene oxide, free-radically polymerizable monomers, for example α,β-unsaturated acids and α,β-unsaturated acid esters. Preferably, the block A is a poly(meth)acrylate, polylactone, polyphenylene oxide or polyalkylene oxide oligomer. Very preferably, the A block is a polymerizate of caprolactone, 2,6-dialkyl-1,4-phenylene oxide and in particular of 2,6-dimethyl-1,4-phenylene oxide, ethylene oxide, propylene oxide or (meth)acrylates. The A block is thus preferably a polycaprolactone (PCL), poly(2,6-dimethyl-1,4-phenylene oxide), poly(ethylene oxide), poly(propylene oxide) or poly(meth)acrylate oligomer.

The B-block is preferably a polysiloxane and/or a polyvinyl and/or a polyalkene and/or a polydiene oligomer or a hydrogenated polydiene oligomer. Particularly preferably, the B-block is a polydiene oligomer or a hydrogenated polydiene oligomer, polyvinyl alkanoate oligomer or a polysiloxane oligomer according to the formula:

—O—(SiR¹²₂—O)ₚ—

in which
R¹² is a linear C₁-C₂₀ alkyl, branched C₃-C₁₂ alkyl or C₆-C₂₀ aryl group, wherein the individual R¹² radicals can be the same or different, and
p is a number from 3 to 100, preferably a number from 10 to 50.

Very preferably, the B-block is a polymer of dimethylchlorosilane, cyclotri- or cyclotetradimethoxysilane, isoprene, vinyl acetate, isobutene, cis-butadiene or ethylene. The B block is thus preferably a poly(dimethylsiloxane) (PDMS), hydrogenated poly(isoprene), poly(vinyl acetate), hydrogenated poly(isobutene), hydrogenated cis-poly(butadiene) or poly(ethylene) oligomer.

The B blocks are characterized by a relatively high flexibility. Flexible blocks are understood to be blocks formed from monomers whose homopolymers have a glass transition temperature T_{G} below 50° C., preferably below 0° C. and most preferably in the range of −30 to −110° C. Block copolymers with flexible blocks improve the fracture toughness, but impair the flexural strength and the modulus of elasticity of the polymers significantly less than internal plasticizers.

Preferred are ABA and AB block copolymers in which the A block or blocks are preferably each an oligomeric polycaprolactone, poly(2,6-dimethyl-1,4-phenylene oxide), poly (ethylene oxide), poly(propylene oxide), poly(meth)acrylate or poly(meth)acrylate-styrene copolymer building block and the B block is preferably a flexible poly(dimethylsiloxane), poly(isoprene) or hydrogenated poly(isoprene), poly(vinyl acetate), poly(isobutene), cis-poly(butadiene) or hydrogenated cis-poly(butadiene) or a poly(ethylene) building block.

Particularly preferred according to the invention are ABA block copolymers in which the A blocks are each an oligomeric polycaprolactone, poly(meth)acrylate or poly(meth)acrylate-styrene copolymer building block and the B block is a flexible poly(dimethylsiloxane), hydrogenated poly(isoprene) or hydrogenated cis-poly(butadiene) building block.

Very particularly preferred are polyester-polysiloxane block copolymers according to the following general formula:

(PCL)_{q}-b-(PDMS)ᵣ-b-(PCL)_{q}

in which
q is in each case a number from 5 to 40, preferably 10 to 20, and
r is a number from 10 to 100, preferably 30 to 60.

(PCL)_{q} stands for polycaprolactone, which is composed of q caprolactone monomers, and (PDMS)ᵣ stands for poly(dimethylsiloxane), which is composed of r dimethylsiloxane monomers. The letter b stands for block.

Further preferred are poly(meth)acrylate-polysiloxane block copolymers comprising a polymethylmethacrylate residue as the A block and a polysiloxane residue as the B block, wherein the polysiloxane residue is preferably as defined above and is most preferably a poly(dimethylsiloxane) residue.

Particularly preferred are also the ABA triblock copolymers PCL-b-PDMS-b-PCL and PMMA-b-PDMS-b-PMMA with a molar ratio A:B of 0.1 to 5 and with a molar mass of preferably 3 to 25 kDa, particularly preferably 4 to 20 kDa and most preferably 5 to 10 kDa. A preferred block copolymer is PCL-b-PDMS-b-PCL, wherein the PDMS blocks have a molar mass of about 3200 g/mol and the PCL blocks each have a molar mass of about 1600 g/mol. PCL stands for polycaprolactone, PDMS for poly(dimethylsiloxane) and PMMA for polymethyl methacrylate.

Block copolymers can be prepared by the known methods of living or controlled polymerization, e.g. by free radical or ionic (anionic and cationic) polymerization, and also by metal-catalyzed ring-opening polymerization of cyclic monomers, such as caprolactone, with controlled free radical polymerization, living anionic polymerization and ring-opening polymerization being preferred. However, block copolymers can also be obtained by linking end groups of homopolymers. The block copolymers used according to the invention may be di- and triblock copolymers.

AB block copolymers can be prepared, for example, by linking an A block with a terminal OH group by esterification with a B block that has a COOH group. End-group functionalized homopolymer blocks can be prepared relatively easily by the methods of controlled free radical polymerization or by end-capping in anionic polymerization. For example, monomer A is anionically polymerized and an OH group is introduced by endcapping. The OH end group can then be esterified with, for example, α-bromoisobutyric acid. The resulting bromine end group then acts as the starting center for the formation of the B block by ATRP (Atom Transfer Radical Polymerization) of monomer B, initiated by metal complexes of, for example, Cu(I), Ru(I) or Fe(II).

Triblock copolymers can be produced in an analogous way. For example, a B block is produced by anionic polymerization of monomer B via a dianion mechanism. The B middle block formed carries an anion end group on both sides, which initiates the anionic polymerization of monomer A to form the two A blocks (method 1). The esterification of a telechelic B block, which carries a suitable functional group at each end, e.g. an OH group, with two A blocks functionalized only on one side, e.g. with a COOH group, yields ABA triblock copolymers (method 2). Finally, OH-telechelic homopolymers of monomer B can be esterified with α-bromoisobutyric acid. The two bromine end groups formed in this way in homopolymer block B can then be used as the starting center for the formation of the two A blocks by ATRP (method 3).

During the synthesis of the block copolymers, terminal or lateral (meth)acrylate groups that are capable of polymerization can also be introduced. These cause a better integration of the block copolymers into the formed polymer networks through radical copolymerization of the (meth)acrylate groups.

The monomers are preferably chosen so that the A blocks are miscible with the resin matrix, i.e. the mixture of components (a) to (d), and the B block is not miscible with the resin matrix. Miscibility is understood here in the sense of thermodynamics in relation to single phase state. Accordingly, a miscible polymer block is understood to be a polymer block of a monomer whose homopolymer is soluble in the resin matrix such that the blend has a transparency of at least 95%. If, on the other hand, the mixture is cloudy or opaque, i.e. if the transparency is significantly less than 95%, the homopolymer and thus the corresponding polymer block is not miscible with the resin matrix. The transparency is measured with a spectrophotometer on 1 mm thick test specimens polished to a high gloss in transmission (D65) according to ISO 10526:1999, e.g. with a Konika-Minolta spectrophotometer of the type CM-5.

The compositions according to the invention may contain, as fracture toughness modifier, alternatively or preferably in addition to the block copolymers, one or more particulate core-shell polymers. Preferred are polymer particles having a soft core, preferably of polybutadiene, hydrogenated polybutadiene, polyisoprene, hydrogenated polyisoprene, polybutyl acrylate, MMA-butadiene-styrene copolymers (MBS) or polydimethylsiloxane, and a hard shell, preferably of PMMA or an MMA-styrene copolymer.

Soft or flexible polymers are understood to be polymers with a glass transition temperature T_{G} below 50° C., preferably below 0° C. and most preferably in the range of −30 to −110° C. A preferred concrete example is PDMS with a T_{G} of about −110° C. By hard polymers is meant polymers with a glass transition temperature above 50° C. and preferably above 80° C. A preferred concrete example is PMMA with a T_{G} of 105-120° C.

The fracture toughness modifying effect of CSP particles in free-radical di(meth)acrylate polymer networks depends primarily on the type of CSP particles, the particle size, the cross-linking density and the weight ratio of core to shell, which is preferably in a range of 1:1 to 200:1. The cross-linking density is largely determined by the proportion of cross-linking monomers in the particle core. This is preferably in a range from 1 to 10 wt. %, based on the mass of the core. According to the invention, particles with a particle size of 0.20 to 50.0 μm are preferred. When incorporating the CSP particles into the dental material, care must be taken to ensure good dispersion.

The fracture toughness modifier(s) are preferably used in an amount of 1 to 10 wt. %, more preferably in an amount of 2 to 8 wt. % and most preferably 3 to 6 wt. %, based on the total weight of the dental material.

Core-shell polymers have the disadvantage that they can impair the transparency of the compositions, which has a negative effect on the depth of cure during photopolymerization and, additionally, a negative effect on the aesthetics of dental moldings. Therefore, according to the invention, materials are preferred which contain a maximum of 3 wt. % and particularly preferably no core-shell particles.

Unless otherwise stated, the molar mass of oligomers and polymers herein is the number average molar mass Mₙ, determined by gel permeation chromatography (GPC).

Gel permeation chromatography (GPC) is a relative method in which molecules are separated on the basis of their size, more precisely on the basis of their hydrodynamic volume. The absolute molar mass is determined by calibration with known standards.

Preferably, narrowly distributed polystyrene standards are used as calibration standards. These are commercially available. Styrene-divinylbenzene columns are used as separation material and tetrahydrofuran (THF) as eluent. Styrene-divinylbenzene columns are suitable for organic, soluble, synthetic polymers. The measurement is carried out with diluted solutions (0.05 to 0.2 wt. %) of the polymers to be investigated.

Alternatively, the number-average molar mass can be determined with the known methods of freezing point depression (cryoscopy), boiling point elevation (ebullioscopy) or from the depression of the vapor pressure (vapor pressure osmometry). These are absolute methods that do not require calibration standards. Concentration series of 4 to 6 diluted polymer solutions with concentrations of 0.005 to 0.10 mol/kg are examined and then the measured values are extrapolated to a concentration of 0 mol/kg.

According to the invention, compositions are preferred which comprise:
(a) 1 to 30 wt. %, preferably 2 to 25 wt. % and particularly preferably 3 to 20 wt. % of at least one transfer reagent of the formula I,
(b) 1 to 97.9 wt. % of at least one urethane group-containing (meth)acrylate monomer, preferably urethane di(meth)acrylate,
(c) 0 to 20 wt. % of one or more mono(meth)acrylates,
(d) 0 to 50 wt. % of one or more di(meth)acrylates without urethane groups,
(e) 1 to 10 wt. % of at least one fracture toughness modifier,
(f) optionally 10 to 5000 ppm of one or more inhibitors,
(g) 0.1 to 5.0 wt. % of at least one photoinitiator for the radical polymerization,
(h) 0 to 30 wt. % of one or more inorganic or organic fillers,
(i) 0 to 30 wt. % of one or more additives.

All percentages refer to the total mass of the composition unless otherwise stated.

Particularly preferred are compositions which comprise:
(a) 1 to 30 wt. %, preferably 2 to 25 wt. % and particularly preferably 3 to 20 wt. %, of at least one transfer reagent of the formula I,
(b) 1 to 97.9 wt. %, preferably 20 to 95.84 wt. % and particularly preferably 30 to 93.78 wt. % of at least one urethane di(meth)acrylate,
(c) 0 to 20 wt. %, preferably 0 to 10 wt. % and particularly preferably 0 wt. % of one or more mono(meth)acrylates,
(d) 0 to 50 wt. %, preferably 0 to 40 wt. % and particularly preferably 0 to 30 wt. % of one or more di(meth)acrylates without urethane groups,
(e) 1 to 10 wt. %, preferably 2 to 8 wt. % and particularly preferably 3 to 6 wt. % of at least one fracture toughness modifier,
(f) optionally 10 to 5000 ppm, preferably 100 to 4000 ppm and particularly preferably 150 to 1500 ppm of one or more phenolic inhibitors,
(g) 0.1 to 5.0 wt. %, preferably 0.15 to 4.0 wt. % and particularly preferably 0.2 to 3.0 wt. % of at least one photoinitiator for free radical polymerization,
(h) 0 to 30 wt. %, preferably 0 to 20 wt. % and particularly preferably 0 to 10 wt. % of one or more inorganic or organic fillers,
(i) 0 to 30 wt. %, preferably 0 to 25 wt. % and particularly preferably 0 to 20 wt. % of further additives.

The compositions according to the invention may contain up to 20 wt. % of mono(meth)acrylates (c), the mono(meth)acrylate(s) preferably being selected from aliphatic, cycloaliphatic, aromatic, bicyclic and tricyclic mono(meth)acrylates, the total amount of mono(meth)acrylates not exceeding the above mentioned ranges. Particularly preferred are aliphatic mono(meth)acrylates or mono(meth)acrylates having a cycloaliphatic, aromatic, bicyclic or tricyclic group, such as benzyl, tetrahydrofurfuryl or isobornyl (meth)acrylate, 2-[(methoxycarbonyl) amino]ethyl methacrylate, 2-[(propoxy carbonyl) amino]ethyl methacrylate, 2-[(isopropoxycarbonyl)amino]ethyl methacrylate, 2-[(butoxycarbonyl) amino]ethyl methacrylate, 2-[(hexyloxycarbonyl)amino]ethyl methacrylate, 2-[(cyclohexyloxycarbonyl)amino]ethyl methacrylate, 2-[(ethylcarbamoyl) oxy]ethyl methacrylate, 2-[(propylcarbamoyl)oxy]ethyl methacrylate, 2-[(isopropylcarbamoyl)oxy]ethyl methacrylate, 2-[(hexylcarbamoyl)oxy]ethyl methacrylate, 2-[(2-tetrahydrofurfuryloxycarbonyl)amino]ethyl methacrylate, 2-(2-oxo-[1,3]-dioxolan-4-yl methoxycarbonylamino)ethyl methacrylate, methacrylic acid 2-(furan-2-yl methoxycarbonylamino) ethylester, (1,3-dioxolanon-2-on-4-yl)methyl methacrylate, 2-phenoxyethyl (meth)acrylate, 2-(o-biphenyloxy)ethyl (meth)acrylate, 2-hydroxy-3-phenoxypropyl (meth)acrylate, 2-[(benzyloxycarbonyl)amino]ethyl (meth)acrylate, 2-[(benzylcarbamoyl)oxy]ethyl (meth)acrylate, 1-phenoxypropan-2-yl (meth)acrylate and 2-(p-cumylphenoxy)ethyl (meth)acrylate, tricylodecane (meth)acrylate, tricyclodecanemethyl (meth)acrylate, 4,7,7-trimethyl bicyclo [2.2.1]heptanyl (meth)acrylate and mixtures thereof.

According to the invention, low-volatility mono(meth)acrylates (c) are preferred. Low-volatility substances are compounds with an evaporation number greater than 35. The evaporation number (VD) indicates how quickly a substance evaporates at room temperature. It is determined according to DIN 53170. The time it takes for a substance to evaporate completely (evaporation time=VDZ) is related to the time diethyl ether takes to evaporate. The higher the evaporation number, the slower a substance evaporates.

Very particularly preferred monofunctional monomers are tetrahydrofurfuryl and isobornyl (meth)acrylate, 2-[(benzyloxycarbonyl)amino]ethyl methacrylate, 2-[(ethyl carbamoyl)oxy]ethyl methacrylate, 2-[(benzylcarbamoyl)oxy]ethyl methacrylate, methacrylic acid 2-(furan-2-yl-methoxycarbonylamino)ethyl ester, 2-phenoxyethyl (meth)acrylate, 2-(o-biphenyloxy)ethyl (meth)acrylate, 2-hydroxy-3-phenoxypropyl (meth)acrylate, 1-phenoxypropan-2-yl (meth)acrylate, 2-(p-cumylphenoxy)ethyl (meth)acrylate, tricylodecane (meth)acrylate, tricyclodecanemethyl (meth)acrylate, 4,7,7-trimethylbicyclo[2.2.1]heptanyl (meth)acrylate and mixtures thereof.

Aliphatic, cycloaliphatic, aromatic, bicyclic and tricyclic mono(meth)acrylates (c) are characterized by good radical polymerization ability. In addition, the polymers of these mono(meth)acrylates have comparatively low polymerization shrinkage and good mechanical properties. Due to their relatively high molar mass (150 to 350 g/mol) and their relatively non-polar structure, the mono(meth)acrylates preferred according to the invention also have a low volatility and a comparatively low viscosity. However, mono(meth)acrylates lead to a reduction in network density. The proportion of mono(meth)acrylates is therefore limited to a maximum of 20 wt. % and preferably a maximum of 10 wt. %. Particularly preferred are compositions which do not contain monofunctional monomers.

The compositions according to the invention may contain as component (d) 0 to 50 wt. % of one or more di(meth)acrylates without urethane groups. Preferred di(meth) acrylates (d) are bisphenol A di(meth)acrylate, bis-GMA (an addition product of (meth)acrylic acid and bisphenol A diglycidyl ether), ethoxy- or propoxylated bisphenol A di(meth)acrylate or 2,2-bis[4-(2-(meth)acryloyloxypropoxy)phenyl]propan, tri- or tetra ethylene glycol di(meth)acrylate, 1,10-decanediol di(meth)acrylate, bis[(meth)acryloyloxymethyl]tricyclo-[5.2.1.0^{2,6}]decane or 1,12-dodecanediol di(meth)acrylate.

The di(meth)acrylates (d) allow a further adjustment of the crosslink density and an influence on the mechanical properties of the polymers. Particularly preferred di(meth)acrylates (d) are bis-GMA, bisphenol A di(meth)acrylate with 3 ethoxy groups, 2,2-bis[4-(2-(meth)acryloyloxypropoxy)phenyl]propane, triethylene glycol di(meth)acrylate, as well as glycerol tri(meth)acrylate, 1,10-decanediol di(meth)acrylate, bis[(meth)acryloyloxymethyl]tricyclo-[5.2.1.0^{2,6}]decane, 1,12-dodecanediol di(meth)acrylate and mixtures thereof.

The preferred di(meth)acrylate monomers (d) are characterized by a relatively low molecular weight, di(meth)acrylates (d) with a molecular weight in the range of 200 to 800 g/mol and in particular 220 to 650 g/mol being particularly preferred. Due to the low molecular weight, in particular in comparison with the urethane di(meth)acrylate telechels (b), the di(meth)acrylate monomers (d) cause a relatively strong crosslinking of the polymers. The proportion of further di(meth)acrylates (d) is therefore limited to a maximum of 50 wt. %, preferably a maximum of 30 wt. %. According to a particularly preferred embodiment, the dental materials according to the invention exclusively contain urethane group-containing (meth)acrylate monomers (b), preferably urethane di(meth)acrylates, as crosslinking agents. Furthermore, it is preferred that the compositions according to the invention do not contain any further polyfunctional monomers, such as tri-, tetra- or higher-functional (meth)acrylates, in addition to the urethane group-containing (meth)acrylate monomers (b) and the di(meth)acrylates (d).

Urethane group-containing (meth)acrylate monomers (b), monofunctional methacrylates (c), and di(meth) acrylate monomers (d) are preferably used in a ratio such that polymers with a network density of v_{c}=800 to 16,000 mol/m³, preferably 2000 to 10,000 mol/m³, particularly preferably 2500 to 8000 mol/m³ are obtained. According to the invention, the crosslinking density is mainly adjusted by the content of transfer reagent (a). In addition, the crosslink density is also influenced by the ratio of crosslinking to monofunctional monomers. Preferred dental materials according to the invention are those in which the mole fraction of the crosslinking monomers is in a range of 0.80 to 1.0. Particularly preferred are resins that do not contain monofunctional monomers.

All radically polymerizable components of the materials according to the invention are used to calculate the mole fraction, i.e. in particular components (b), (c) and (d). Cross-linking monomers are understood to be all radically polymerizable components which have two or more radically polymerizable groups, i.e. in particular components (b) and (d). Cross-linking monomers are also referred to as polyfunctional monomers. Monofunctional monomers are monomers with only one radical polymerizable group.

The network density corresponds to the number of network nodes (in mol) per unit volume and can be calculated by dynamic-mechanical measurements from the plateau value of the storage modulus G′ in the elastic range. The glass transition temperature T_{G} and the network density v_{c} are determined with a rheometer, preferably an Anton Paar Rheometer MCR301. For this purpose, storage and loss modulus of a test specimen (25×5×1 mm, clamped longitudinally) are measured between 25° C. and 250° C. (frequency 1 Hz, deformation 0.05%, heating rate 2K/min). T_{G} is the maximum of the loss factor tan δ (ratio of loss modulus to storage modulus). The network density is calculated according to the formula v_{c}=G′/(RT), where G′ is the storage modulus at the temperature T_{G}+50 K, R is the general gas constant, and T is the temperature at T_{G}+50 K in Kelvin.

The compositions according to the invention preferably contain at least one inhibitor (f), more preferably in an amount of from 10 to 5000 ppm, most preferably in an amount of from 100 to 4000 ppm and most preferably from 150 to 1500 ppm, based on the total mass of the composition. Inhibitors are understood to be free radical scavenging agents to prevent premature polyreaction. The inhibitors are also referred to as polymerization inhibitors. They improve the storage stability of the materials. Preferred inhibitors are phenolic inhibitors, in particular hydroquinone monomethyl ether (MEHQ, CAS: 150-76-5), 2,6-di-tert-butyl-4-methyl phenol (BHT, CAS: 128-37-0), 2,6-di-tert-butyl phenol (CAS: 128-39-2), pyrogallol (CAS: 87-66-1), hydroquinone (CAS: 123-31-9), 2-tert-butylhydroquinone (CAS: 1948-33-0), 4-tert-butyl catechol (CAS: 98-29-3) and 6-tert-butyl-2,4-xylenol (CAS: 1879-09-0), MEHQ, pyrogallol and BHT being particularly preferred.

It was surprisingly found that the preferred phenolic inhibitors, especially in an amount of 150 to 1500 ppm, protect the compositions according to the invention very well against premature polymerization on the one hand and ensure good polymerization reactivity on the other hand. After curing, they lead to materials with high flexural strength, high flexural modulus and high work of rupture.

The compositions according to the invention contain at least one photoinitiator (g). Photoinitiators for the UV or preferably the visible wavelength range of light can be used.

Preferred photoinitiators for the visible range are α-diketones and their derivatives, such as 9,10-phenanthrenequinone, 1-phenyl-propane-1,2-dione, diacetyl or 4,4′-dichlorobenzil. Particularly preferred are camphorquinone (CQ) and 2,2-dimethoxy-2-phenyl-acetophenone and most preferred α-diketones in combination with amines as reducing agents, such as 4-(dimethylamino)-benzoic acid ester (EDMAB), N,N-dimethylaminoethyl methacrylate, N,N-dimethyl-3,5-xylidine or triethanolamine. Other preferred photoinitiators for the visible range are monomolecular photoinitiators, so-called Norrish type I photoinitiators, in particular monoacyltrialkyl-, diacyldialkyl- and tetraacylgermanium as well as tetraacylstannanes, such as benzoyltrimethylgermanium, dibenzoyldiethylgermanium, bis(4-methoxybenzoyl)diethylgermanium, tetrakis(2-methylbenzoyl)germanium or tetrakis(mesitoyl)stannane. Mixtures of the various photoinitiators can also be used, such as bis(4-methoxy benzoyl) diethylgermanium in combination with camphorquinone and 4-dimethylamino benzoic acid ethyl ester.

Particularly preferred photoinitiators for the UV and visible range are monoacyl phosphines such as diphenyl-(2,4,6-trimethylbenzoyl)phosphine oxide (Lucirin® TPO, CAS: 75980-60-8) or 2,4,6-trimethylbenzoylethoxylphenylphosphine oxide (Lucirin® TPO-L CAS: 84434-11-7) or other 2,4,6-trimethylbenzoylalkoxyphenylphosphine oxides and further bisacylphosphine oxides, such as Irgacure 819 (bis(2,4,6-trimethylbenzoyl)phenylphosphine oxide) (CAS: 16881-26-7). Particularly preferred photoinitiators are diphenyl(2,4,6-trimethylbenzoyl)phosphine oxide and 2,4,6-trimethylbenzoylethoxylphenylphosphine oxide.

Preferred UV photoinitiators are acetophenones, e.g. 2,2-diethoxy-1-phenylethanone, benzoin ether, such as Irgacure 651 (dimethylbenzilketal), hydroxyalkylphenylacetophenone, such as Irgacure 184 (1-hydroxy-cyclohexyl-phenyl-ketone), 2-benzyl-2-(dimethylamino)-4′-morpholinobutyrophenone (Irgacure 369) and 1-butanone-2-(dimethylamino)-2-(4-methylphenyl)methyl-1-4-(4-morpholinyl)phenyl (Irgacure 379).

For post-treatment, it is advantageous to use two photoinitiators that differ in their absorption ranges. Mixtures of photoinitiators for the UV range and the visible range are preferred. Post-tempering of free-radical polymerizates means additional irradiation of the polymerizates and/or subsequent heat treatment to increase the double bond or monomer conversion and thus to improve the mechanical and optical properties of the polymers.

The dental materials according to the invention may additionally contain one or more thermal initiators, e.g. azo compounds, such as 2,2′-azobis(isobutyronitrile) (AIBN) or azobis-(4-cyanovaleric acid), or peroxides, such as dibenzoyl peroxide, dilauroyl peroxide, tert-butyl peroctoate, tert-butyl perbenzoate or di-(tert-butyl) peroxide. Combinations with aromatic amines can also be used to accelerate initiation by peroxides. Preferred redox systems are combinations of dibenzoyl peroxide with amines, such as N,N-dimethyl-p-toluidine, N,N-dihydroxyethyl-p-toluidine, p-dimethylaminobenzoic acid ethyl ester or structurally related systems.

The compositions according to the invention may further contain one or more fillers (H), preferably particulate or fibrous inorganic or organic fillers or composite fillers. Fillers allow the mechanical properties to be influenced. Particularly preferred are inorganic particulate fillers.

Preferred inorganic fillers are oxides, such as SiO₂, ZrO₂ and TiO₂ or mixed oxides of SiO₂, ZrO₂, ZnO and/or TiO₂, nanoparticulate or microfine fillers, such as fumed silica or precipitated silica, glass powders, such as quartz, glass-ceramic, borosilicate or radiopaque glass powders, preferably barium or strontium aluminosilicate glasses, and radiopaque fillers, such as ytterbium trifluoride, tantalum(V) oxide, barium sulphate or mixed oxides of SiO₂ with ytterbium(III) oxide or tantalum(V) oxide. Furthermore, the dental materials according to the invention may contain fibrous fillers, nanofibres, whiskers or mixtures thereof. According to a preferred embodiment, the materials according to the invention do not contain fluoroaluminosilicate glasses, calcium aluminosilicate glasses or other fillers which react with organic acids in the sense of an acid-base reaction.

Preferably, the oxides have a particle size of 0.010 to 15 μm, the nanoparticulate or microfine fillers have a particle size of 10 to 300 nm, the glass powders have a particle size of 0.10 to 15 μm, preferably of 0.2 to 1.5 μm, and the radiopaque fillers have a particle size of 0.2 to 5 μm.

Particularly preferred fillers are mixed oxides of SiO₂ and ZrO₂ with a particle size of 10 to 300 nm, glass powders with a particle size of 0.2 to 1.5 μm, in particular X-ray opaque glass powders e.g. of barium or strontium aluminosilicate glasses, and X-ray opaque fillers with a particle size of 0.2 to 5 μm, in particular ytterbium trifluoride and/or mixed oxides of SiO₂ with ytterbium(III) oxide.

To improve the bond between the filler particles and the crosslinked polymerization matrix, SiO₂-based fillers can be surface-modified with methacrylate-functional silanes. A preferred example of such silanes is 3-methacryloyloxypropyltrimethoxysilane. The surfaces of non-silicate fillers such as ZrO₂ or TiO₂ can also be modified with functionalized acid phosphates such as 10-methacryloyloxydecyl dihydrogen phosphate.

Other preferred fillers are particulate waxes, especially carnauba wax, preferably with a particle size of 1 to 10 μm, non-crosslinked or partially crosslinked polymethyl methacrylate (PMMA) particles, preferably with a particle size of 500 nm to 10 μm, and polyamide-12 particles, preferably with a particle size of 5 to 10 μm.

Furthermore, the dental materials according to the invention may contain so-called composite fillers or iso-fillers, i.e. ground composites which preferably have a broad particle size distribution, e.g. with particle sizes of 0.05 to 20 μm, in particular about 0.1 to about 10 μm. Preferably, the composite filler or isofiller is surface-modified, in particular salinized.

Unless otherwise stated, all particle sizes herein are weight-average particle sizes, and determination of particle size in the range of 0.1 μm to 1000 μm is performed by static light scattering, preferably using a static laser scattering particle size analyzer LA-960 (Horiba, Japan). Here, a laser diode with a wavelength of 655 nm and an LED with a wavelength of 405 nm are used as light sources. The use of two light sources with different wavelengths enables the measurement of the entire particle size distribution of a sample in only one measurement run, whereby the measurement is carried out as a wet measurement. For this, a 0.1 to 0.5% aqueous dispersion of the filler is prepared and its scattered light is measured in a flow cell. The scattered light analysis for calculating particle size and particle size distribution is carried out according to the Mie theory as per DIN/ISO 13320.

Particle sizes smaller than 0.1 μm are preferably determined by dynamic light scattering (DLS). The measurement of particle size in the range of 5 nm to 0.1 μm is preferably carried out by Dynamic Light Scattering (DLS) of aqueous particle dispersions, preferably with a Malvern Zetasizer Nano ZS (Malvern Instruments, Malvern UK) with a He—Ne laser with a wavelength of 633 nm, at a scattering angle of 90° at 25° C.

The light scattering decreases with decreasing particle size. Particle sizes smaller than 0.1 μm can also be determined by SEM or TEM spectroscopy. Transmission electron microscopy (TEM) is preferably performed with a Philips CM30 TEM at an accelerating voltage of 300 kV. For sample preparation, drops of the particle dispersion are applied to a 50 Å thick copper grid (mesh size: 300 mesh), which is coated with carbon, and the solvent is subsequently evaporated.

The fillers are subdivided according to their particle size into macrofillers and microfillers, whereby fillers with an average particle size of 0.2 to 10 μm are called macrofillers and fillers with an average particle size of approx. 5 to 100 nm are called microfillers. Macrofillers are obtained by grinding e.g. quartz, radiopaque glasses, borosilicates or ceramics and usually consist of splinter-shaped particles. Fumed SiO₂ or precipitated silica or mixed oxides, e.g. SiO₂—ZrO₂, which are accessible by hydrolytic co-condensation of metal alkoxides, are preferably used as microfillers. The microfillers preferably have an average particle size of about 5 to 100 nm. Fillers with a small particle size have a greater thickening effect.

In a preferred embodiment, the compositions according to the invention contain a mixture of two or more fillers, in particular two or more fillers with different particle sizes. It has been found that the use of such mixtures of fillers does not excessively increase the viscosity of the materials and therefore the compositions are readily processable by generative methods, such as by stereolithography. The total content of fillers is preferably in a range from 0 to 30 wt. %, particularly preferably from 0 to 10 wt. %.

The compositions according to the invention may further contain one or more additives (i), in particular UV absorbers, optical brighteners, colorants, plasticizers and/or thixotropic additives.

The compositions may contain one or more UV absorbers. The UV absorber serves to reduce the penetration depth of the light and thus the polymerization depth during the light-induced curing of the composition according to the invention. This proves to be particularly advantageous in stereolithographic applications, since only thin layers are to be cured in stereolithography. By using a UV absorber, precision can be improved in stereolithographic processes.

Preferred UV absorbers are those based on benzotriazole, benzophenone or triazines. Particularly preferred UV absorbers are 2,2′-methylenebis[6-(2H-benzotriazol-2-yl)-4-(1,1,3,3-tetramethylbutyl)phenol], 2,2′,4,4′-tetrahydroxybenzophenone, 2-tert-butyl-6-(5-chloro-2H-benzotriazol-2-yl)-4-methylphenol (bumetrizole), 2,2′-benzene-1,4-diylbis(4h-3,1-benzoxazin-4-one), 2-(4,6-bis-(2,4-dimethylphenyl)-1,3,5-triazin-2-yl)-5-(octyloxy)-phenol, 2-(2-hydroxy-5-methylphenyl)benzotriazole, 2-(2-hydroxyphenyl) benzotriazole, 2-(2H-benzotriazol-2-yl)-6-dodecyl-4-methylphenol, 2-(2′-hydroxy-3′, 5′-di-t-butylphenyl)-5-chlorobenzotriazoles, 2,2′-dihydroxy-4-methoxybenzophenone and 2,2′-dihydroxy-4,4′-dimethoxybenzophenone. Further preferred are so-called hindered amines light stabilizers such as bis(1,2,2,6,6-pentamethyl-4-piperidyl)sebacate, methyl-1,2,2,6,6-pentamethyl-4-piperidylsebacate, bis(1-octyloxy-2,2,6,6-tetramethyl-4-piperidyl)sebacate and bis(1,2,2,6,6-pentamethyl-4-piperidyl)-[[3,5-bis(1,1-dimethylethyl)-4-hydroxyphenyl]methyl]butylmalonate. Very particularly preferred UV absorbers are bumetrizole and 2,2′,4,4′-tetrahydroxybenzophenone.

The UV absorber preferably has an absorption maximum that corresponds to the wavelength of the light used for curing. UV absorbers with an absorption maximum in the range of 320 to 500 nm and preferably 380 to 480 nm are advantageous, whereby UV absorbers with an absorption maximum below 400 nm are particularly preferred.

UV absorbers are optionally used in an amount preferably up to 0.5 wt. %. Bumetrizole is preferably used in an amount up to 0.15 wt. %, and 2,2′,4,4′-tetrahydroxybenzophenone in an amount up to 0.07 wt. %. All data refer to the total weight of the material. Dental materials that do not contain a UV absorber are particularly preferred.

The compositions according to the invention may contain one or more optical brighteners. Preferred optical brighteners according to the invention are those that absorb light in the UV range, i.e. light with a wavelength below 400 nm. By adding an optical brightener, the penetration depth of the light and thus the curing depth can be reduced, thus increasing the precision in stereolithographic processes. Optical brighteners that are capable of re-emitting the light absorbed in the UV range as light with a wavelength of 400 to 450 nm are particularly preferred. Such optical brighteners increase the reactivity of the materials by emitting the absorbed short-wave light as longer-wave blue light due to their fluorescence, thus providing additional light power for photoinitiation. Optical brighteners preferred according to the invention are 2,5-bis(5-tert-butyl-benzoxazol-2-yl) thiophene and fluorescent agents in the form of terephthalic acid derivatives, such as 2,5-dihydroxyterephthalic acid diethyl ester or diethyl 2,5-dihydroxyterephthalate.

The optical brightener(s) may be used in an amount of preferably up to 0.05 wt. %, based on the total weight of the dental material. Compositions containing no optical brightener are particularly preferred.

Optical brighteners can be used in combination with UV absorbers. In this case, it is preferred that the weight ratio of UV absorber to optical brightener is in the range of 2:1 to 50:1, more preferably 2:1 to 30:1 and most preferably 2:1 to 5:1 or 10:1 to 25:1. Preferred combinations are those containing 2,2′,4,4′-tetrahydroxybenzophenone or bumetrizole as UV absorbers and 2,5-bis(5-tert-butyl-benzoxazol-2-yl)thiophene as optical brightener. Very particularly preferred is the combination of 2,2′,4,4′-tetrahydroxybenzophenone and 2,5-bis(5-tert-butyl-benzoxazol-2-yl)thiophene in a weight ratio of 2:1 to 10:1, preferably 2:1 to 5:1, or the combination of bumetrizole and 2,5-bis(5-tert-butyl-benzoxazol-2-yl)thiophene in a weight ratio of 5:1 to 30:1, preferably 10:1 to 20:1.

The compositions according to the invention may contain one or more colorants, preferably in a concentration of 0 to 0.5 wt. %. The colorants serve primarily aesthetic purposes. Colorants preferred according to the invention are organic dyes and pigments, in particular azo dyes, carbonyl dyes, cyanine dyes, azomethines and methines, phthalocyanines and dioxazines. Particularly preferred are dyes which are soluble in the materials of the invention, especially azo dyes. Also suitable as colorants are inorganic and in particular organic pigments which are readily dispersible in the dental materials according to the invention. Preferred inorganic pigments are metal oxides or hydroxides, such as titanium dioxide or ZnO as white pigments, iron oxide (Fe₂O₃) as red pigment or iron hydroxide (FeOOH) as yellow pigment. Preferred organic pigments are azo pigments, such as monoazo yellow and orange pigments, diazo pigments or β-naphthol pigments, and non-azo or polycyclic pigments, such as phthalocyanine, quinacridone, perylene and flavanthron pigments. Azo pigments and non azo pigments are particularly preferred.

Furthermore, compositions according to the invention may contain one or more plasticizers. Plasticizers prevent the polymers from becoming brittle after photochemical curing and possible drying. In addition, plasticizers provide sufficient flexibility. Plasticizers are preferably added in a concentration of 0.2 to 5 wt. %. Preferred plasticizers are phthalates, such as dibutyl or dihexyl phthalate, non-acidic phosphates, such as tributyl or tricresyl phosphate, n-octanol, glycerol or poly ethylene glycols. Particularly preferred are tartaric acid or citric acid esters, such as citric acid triester, which are characterized by good biocompatibility.

The compositions according to the invention can also contain one or more thixotropic agents. These additives cause the materials to thicken and can thus, for example, prevent sedimentation of the fillers. In particular, filler-containing compositions therefore preferably contain at least one thixotropy additive. Preferred thixotropic agents are polymers containing OH groups, such as cellulose derivatives, and inorganic substances, such as layered silicates. In order not to increase the viscosity of the materials too much, the dental materials according to the invention preferably contain only 0 to 3.0 wt. %, particularly preferably 0 to 2.0 wt. % and most preferably 0.1 to 2.0 wt. % of thixotropic additive, based on the total weight of the composition.

Certain fillers, such as highly dispersed SiO₂, i.e. SiO₂ with small primary particle size (<20 nm) and high specific surface area (>100 m²/g) also have a thixotropic effect. Such fillers can replace thixotropic additives.

The rheological properties of the compositions according to the invention are adapted to the desired application. Materials for stereolithographic processing are preferably adjusted so that their viscosity is in the range of 50 mPa-s to 100 Pa-s, preferably 100 mPa-s to 10 Pa-s, particularly preferably 100 mPa-s to 5 Pa-s. The viscosity is determined at 25° C. using a cone-plate viscometer (shear rate 100/s). Particularly preferably, the compositions according to the invention have a viscosity<10 Pa-s and more preferably <5 Pa-s at 25° C. The viscosity is preferably determined using an Anton Paar viscometers of the type MCR 302 with CP25-2 cone-plate measuring means and a measuring gap of 53 μm in rotation at a shear rate of 100/s. Due to the low viscosity, the compositions according to the invention are particularly suitable to be processed by generative manufacturing methods, such as 3D printing or stereolithography. The processing temperature is preferably in a range from 10 to 70° C., particularly preferably 20 to 30° C.

According to the invention, it was surprisingly found that by combining fracture toughness modifiers with a transfer reagent of formula I, a significant improvement in fracture toughness is possible even without the use of monofunctional monomers. This applies in particular to the combination with the fracture toughness modifiers preferred according to the invention. In this way, the amount of monofunctional monomers can be reduced or their addition can be completely eliminated, thus avoiding the associated disadvantages. The compositions according to the invention are further characterized by the fact that, in addition to high fracture toughness and fracture work, they simultaneously have good flexural strength and a relatively high modulus of elasticity, measured after storage at 37° C. in water, which corresponds to oral conditions. The materials also have a low viscosity. Furthermore, it is particularly advantageous that the dental materials have a low inherent color even after curing.

After hardening, the materials according to the invention have a flexural strength of greater than 60 MPa, preferably greater than 70 MPa, particularly preferably greater than 75 MPa, a flexural modulus of greater than 1.50 GPa, preferably greater than 1.70 GPa, particularly preferably greater than 2.00 GPa, each measured according to ISO 4049, and a fracture work FW greater than 180 J/m², preferably greater than 200 J/m², particularly preferably greater than 210 J/m², measured according to ISO 20795-1:2013. Workpieces made from these materials are thus highly resistant to deformation without fracture. High transparency combined with high fracture toughness cannot be achieved with core-shell polymers.

Due to the above properties, compositions according to the invention are excellently suited as dental materials and in particular for the manufacture or repair of dental moldings. The invention also relates to the use of the compositions according to the invention as dental materials and in particular for the manufacture or repair of dental workpieces, such as dental restorations, prostheses, prosthetic materials, artificial teeth, inlays, onlays, crowns, bridges, drilling templates, trial bodies or orthodontic devices.

In addition, the present invention also relates to a process for the production of dental moldings, in particular for the production of the above-mentioned dental moldings, in which a composition according to the invention is cured with the aid of light to yield the dental molding. The invention also relates to dental moldings, in particular the above-mentioned moldings, obtainable by such a process.

The fabrication or repair of dental moldings is preferably carried out extraorally. Furthermore, it is preferred that the production or repair of dental moldings is carried out by a generative process, in particular by means of 3D printing or a lithography-based process, such as stereolithography.

For this, a virtual image of the tooth situation is created by directly or indirectly digitizing the tooth or teeth to be restored on the computer, then a model of the dental restoration or prosthesis is constructed on the computer on the basis of this image and this model is then produced by generative stereolithographic production. Once a virtual model of the dental restoration or prosthesis to be produced has been created, the composition according to the invention is polymerized by selective irradiation with light. The geometry of the dental restoration or prosthesis can be built up layer by layer by successively polymerizing a plurality of thin layers with a desired cross-section. The layer-by-layer build-up of the geometry is preferably followed by a cleaning of the workpiece by treatment with a suitable solvent, e.g, an alcohol, such as ethanol or isopropanol, a ketone or an ester, and a post-treatment by irradiation of the workpiece with a suitable wavelength, such as irradiation with an intensity of e.g. 25 mW/cm² at 405 nm and simultaneously 130 mW at 460 nm for 15 min, optionally with simultaneous heating to 50° C. or more, in order to further reduce the residual monomer content and improve the mechanical properties.

The invention is explained in more detail below with the aid of examples.

### EXAMPLES

### Example 1

### Preparation of the ABA Block Copolymer BC-1 (PCL(800)-b-PDMS(1600)-b-PCL(800) Block Copolymer)

A mixture of bisaminopropyl-terminated polydimethylsiloxane (PDMS from Gelest) with a molar mass of 1600 g/mol (20.65 g) and 8-caprolactone (CL: 21.10 g) was heated to 80° C. After 1 h, tin bis(2-ethylhexanoate) (10 mg) was added and the bath temperature was gradually increased to 130° C. within 30 min. The reaction mixture was stirred for a further 22 h at 130° C. Subsequently, volatile components were distilled off at fine vacuum. 40.47 g (97%) PCL(800)-b-PDMS(1600)-b-PCL(800) block copolymer BC-1 was obtained as a waxy, slightly brownish solid.

¹H NMR (CDCl₃, 400 MHz): 5=4.14 (t, 26H; J=6.7 Hz; O—CH₂), 3.71 (t, 4H; J=6.4 Hz; HO—CH₂), 3.30 (q, 4H; J=6.4 Hz; N—CH₂), 2.39 (t, 26H; J=7.5 Hz; C(O)—CH₂), 2.24 (t, 4H; J=7.5 Hz; N—CH₂), 1.78-1.66 (m, 56H; CH₂), 1.53-1.36 (m, 30H; CH₂), 0.65-0.56 (m, 4H; Si—CH₂), 0.15 (s, 126H; Si—CH₃).

### Example 2

Preparation and Curing of Compositions with the Urethane Dimethacrylate UDMA, the Diluent DEGDMA and the Transfer Reagent TSMAE in the Molar Ratio 7.2/1.8/1.0 and Different Amounts of ABA Block Copolymer BC-1

The monomer components urethane dimethacrylate UDMA (an addition product of 2 mol 2-hydroxyethyl methacrylate and 1 mol 2,2,4-trimethylhexamethylene-1,6-diisocyanate) and diethylene glycol dimethacrylate (DEGDMA) as well as the transfer reagent 2-toluene-4-sulfonylmethyl)acrylic acid ethyl ester (TSMAE) were mixed in the molar ratio 7.2 mol UDMA/1.8 mol DEGDMA/1.0 mol TSMAE and were then mixed with 1.5 mol-% of the photoinitiator diphenyl-(2,4,6-trimethylbenzoyl)phosphine oxide (TPO). The obtained monomer mixture was homogeneously mixed with 3 wt. % (monomer resin R1), 5 wt. % (monomer resin R2) or 10 wt. % (monomer resin R3) of the block copolymer BC-1. For this, the components were dissolved with a planetary mixer under stirring and further stirred until a homogeneous mixture was achieved. Completely transparent resins were obtained in all cases. The compositions of the resin mixtures are given in Table 1.

TABLE 1
Composition of the resins R1-R3 (masses in wt.-%)

| Monomer resin | UDMA^{b)} | DEGDMA^{d)} | TSMAE^{a)} | TPO^{c)} | BC-1 |
|---|---|---|---|---|---|
| R1 | 79.22 | 10.24 | 6.32 | 1.22 | 3.00 |
| R2 | 77.59 | 10.03 | 6.18 | 1.20 | 5.00 |
| R3 | 73.51 | 9.49 | 5.86 | 1.14 | 10.00 |

^{a)}TSMAE: 2-(toluene-4-sulfonylmethyl)-acrylic acid ethyl ester
^{b)}UDMA: an addition product of 2 mol 2-hydroxyethyl methacrylate and 1 mol 2,2,4-trimethylhexamethylene-1,6-diisocyanate
^{c)}Diphenyl-(2,4,6-trimethylbenzoyl)phosphine oxide (photoinitiator)
^{d)}Diethylene glycol dimethacrylate

The resins were used to prepare test specimens in metal molds, which were irradiated on both sides with a dental light source (PrograPrint Cure, Ivoclar Vivadent AG, Schaan, Liechtenstein; software: ProArt Print Splint, year: 2020) for 2×1.5 minutes and thus cured.

Further processing and storage of the test specimens was carried out according to the relevant specifications of the regulations mentioned below. The flexural strength (BF) and flexural modulus of elasticity (BM) were determined according to the ISO standard ISO-4049 (Dentistry—Polymer-based filling, restorative and luting materials). For this, the test specimens were either stored dry for 24 h at room temperature (RT) or for 24 h at 37° C. in water (H₂O) before measurement. The determination of the fracture toughness Kₘₐₓ and the fracture work FW were carried out according to ISO 20795-1:2013 (Dentistry—Plastics—Part 1: Denture resins). The results of the measurements are summarized in table 2.

TABLE 2
Mechanical properties of the polymerized resins R1-R3

| | BF 24 h, | BF 24 h, 37° C. | BM 24 h, | BM 24 h, 37° C. | Kₘₐₓ | FW |
|---|---|---|---|---|---|---|
| Resin | RT (MPa) | H₂O (MPa) | RT (MPa) | H₂O (MPa) | (MPa · m^{0.5}) | (J/m²) |
| R1 | 102.9 ± 1.8 | 102.6 ± 1.3 | 2615 ± 109 | 2574 ± 98 | 1.35 ± 0.05 | 181 7± |
| R2 | 95.4 ± 4.9 | 88.3 ± 7.6 | 2537 ± 66 | 2392 ± 83 | 1.41 ± 0.02 | 204 ± 12 |
| R3 | 81.1 ± 1.5 | 77.5 ± 5.4 | 2154 ± 26 | 2033 ± 40 | 1.50 ± 0.04 | 289 ± 13 |

The results prove that the combination of transfer reagent and block copolymer yields photopolymerizates with good fracture toughness and flexural strength even without monofunctional monomer.

### Example 3

Preparation and Curing of Compositions with the Urethane Dimethacrylate UDMA, the Diluent DEGDMA and the Transfer Reagent TSMAE in the Molar Ratio 6.4/1.6/2.0 and Different Amounts of ABA Block Copolymer BC-1

Analogous to example 2, the monomer components UDMA and DEGDMA as well as the transfer reagent TSMAE were mixed in a molar ratio of 6.4 mol UDMA/1.6 mol DEGDMA/2.0 mol TSMAE and then mixed with 1.5 mol-% of the photoinitiator TPO. The obtained monomer mixture (monomer resin R4) was homogeneously mixed with 3 wt. % (monomer resin R5), 5 w.t % (monomer resin R6) or 10 wt. % (monomer resin R7) of the block copolymer BC-1. Test specimens were then prepared and the mechanical properties measured. The compositions of the resin mixtures are summarized in Table 3 and the results of the measurements in Table 4.

TABLE 3
Composition of the resins R4-R7 (masses in wt.-%)

| Monomer resin | UDMA^{b)} | DEGDMA^{d)} | TSMAE^{a)} | TPO^{c)} | BC-1 |
|---|---|---|---|---|---|
| R4*) | 75.57 | 9.68 | 13.43 | 1.31 | — |
| R5 | 73.30 | 9.39 | 13.04 | 1.27 | 3.00 |
| R6 | 71.79 | 9.20 | 12.76 | 1.25 | 5.00 |
| R7 | 68.01 | 8.71 | 12.10 | 1.18 | 10.00 |

*)Comparative example
^{a)-d)}as table 1

TABLE 4
Mechanical properties of polymerized resins R4-R7

| | BF 24 h, | BF 24 h, 37° C. | BM 24 h, | BM 24 h, 37° C. | Kₘₐₓ | FW |
|---|---|---|---|---|---|---|
| Resin | RT (MPa) | H₂O(MPa) | RT (MPa) | H₂O (MPa) | (MPa · m^{0.5}) | (J/m²) |
| R4*) | 100.3 ± 6.3 | 102.8 ± 6.9 | 2677 ± 61 | 2642 ± 107 | 0.99 ± 0.05 | 95 9± |
| R5 | 90.7 ± 2.7 | 95.1 ± 5.5 | 2479 ± 90 | 2568 ± 123 | 1.91 ± 0.07 | 376 ± 18 |
| R6 | 95.5 ± 6.3 | 93.4 ± 6.8 | 2525 ± 45 | 2356 ± 84 | 1.71 ± 0.06 | 339 ± 31 |
| R7 | 69.9 ± 1.4 | 72.4 ± 3.0 | 2050 ± 26 | 2099 ± 40 | 1.85 ± 0.04 | 534 ± 11 |

*)Comparative example

Example 3 shows in comparison with example 2 that the fracture toughness, i.e. Kmax and the fracture work, of the photopolymers increase very significantly with increasing concentration of the transfer reagent without a significant reduction in the flexural strength or the flexural modulus. The results prove that by using transfer reagents in combination with block copolymers, photopolymerizates with good fracture toughness can be obtained even without monofunctional monomers.

### Example 4

Preparation and Curing of Compositions with the Urethane Dimethacrylate UDMA, the Diluent DEGDMA and the Transfer Reagent TSMAE in the Molar Ratio 5.6/1.4/3.0 and Different Amounts of ABA Block Copolymer BC-1

Analogous to example 2, the monomer components UDMA and DEGDMA as well as the transfer reagent TSMAE were mixed in a molar ratio of 5.6 mol UDMA/1.4 mol DEGDMA/3.0 mol TSMAE and then mixed with 1.5 mol-% of the photoinitiator TPO. The obtained monomer mixture was homogeneously mixed with 3 wt. % (monomer resin R8), 5 wt. % (monomer resin R9) or 10 wt. % (monomer resin R10) of the block copolymer BC-1. Subsequently, test specimens were prepared and the mechanical properties were measured. The compositions of the resin mixtures are summarized in Table 5 and the results of the measurements in Table 6.

TABLE 5
Composition of the resins R8-R10 (masses in wt.-%)

| Monomer resin | UDMA^{b)} | DEGDMA^{d)} | TSMAE^{a)} | TPO^{c)} | BC-1 |
|---|---|---|---|---|---|
| R8 | 66.69 | 8.61 | 20.38 | 1.32 | 3.00 |
| R9 | 65.31 | 8.44 | 19.96 | 1.29 | 5.00 |
| R10 | 61.88 | 7.99 | 18.90 | 1.23 | 10.00 |

^{a)-d)}as table 1

TABLE 6
Mechanical properties of the polymerised resins R8-R10

| | BF 24 h, | BF 24 h, 37° C. | BM 24 h, | BM 24 h, 37° C. | Kₘₐₓ | FW |
|---|---|---|---|---|---|---|
| Resin | RT (MPa) | H₂O (MPa) | RT (MPa) | H₂O (MPa) | (MPa · m^{0.5}) | (J/m²) |
| R8 | 83.6 ± 0.9 | 79.3 ± 2.3 | 2486 ± 59 | 2295 ± 64 | 2.05 ± 0.07 | 484 ± 32 |
| R9 | 77.7 ± 4.4 | 72.9 ± 1.3 | 2323 ± 35 | 2120 ± 99 | 2.21 ± 0.06 | 617 ± 39 |
| R10 | 65.6 ± 2.4 | 59.4 ± 2.3 | 2071 ± 91 | 1796 ± 75 | 2.06 ± 0.04 | 837 ± 22 |

Example 4 shows, in comparison with examples 2 and 3, that the fracture toughness of the photopolymers can be further increased by increasing the concentration of transfer reagent.

### Example 5

Preparation and Curing of Compositions without Transfer Reagent (Reference Example)

In addition to examples 2 to 4, two comparative examples were carried out, in which a) the resin R11—analogous to R4—comprising only urethane dimethacrylate UDMA and the diluent DEGDMA in the molar ratio 8/2, but no transfer reagent TSMAE and no block copolymer was investigated, and b) a resin R12 comprising urethane dimethacrylate UDMA and the diluent DEGDMA in the molar ratio 8/2 and the block copolymer BC-1 (10 wt. %) but no transfer reagent TSMAE was tested. The resins R11 and R12 also contain 1.5 mol % of the photoinitiator TPO and were prepared as homogeneous mixtures analogous to examples 2 to 4, test specimens were prepared and the mechanical properties were measured. The compositions of the resin mixtures are summarized in Table 7 and the results of the measurements in Table 8.

TABLE 6
Composition of the resins R11-R12 (masses in wt.-%)

| Monomer resin | UDMA^{b)} | DEGDMA^{d)} | TSMAE^{a)} | TPO^{c)} | BC-1 |
|---|---|---|---|---|---|
| R11*) | 87.52 | 11.26 | — | 1.22 | — |
| R12*) | 78.77 | 10.14 | — | 1.09 | 10.00 |

*)Comparative example
^{a)-d)}as table 1

TABLE 8
Mechanical properties of polymerized resins R11-R12

| | BF 24 h, RT | BF 24 h, 37° C. | BM 24 h, | BM 24 h, 37° C. | Kₘₐₓ | FW |
|---|---|---|---|---|---|---|
| Resin | (MPa) | H₂O (MPa) | RT (MPa) | H₂O (MPa) | (MPa · m^{0.5}) | (J/m²) |
| R11 | 119.3 ± 3.3 | 124.5 ± 5.4 | 2871 ± 58 | 2857 ± 75 | 1.18 ± 0.06 | 132 ± 12 |
| R12 | 95.9 ± 2.1 | 92.2 ± 2.3 | 2392 ± 66 | 2164 ± 27 | 1.21 ± 0.04 | 196 ± 15 |

The comparison of the comparative examples R4, R11 and R12 with the examples R1 to R3 and R5 to R10 according to the invention proves that a good fracture toughness (Kₘₐₓ) and a high fracture work (FW) can only be achieved by combining transfer reagent and block copolymer.

1. A radically polymerizable composition comprising
at least one urethan group-containing (meth)acrylate monomer,
at least one fracture toughness modifier and
at least one transfer agent,
characterised in that the transfer agent is a compound of the general formula I: 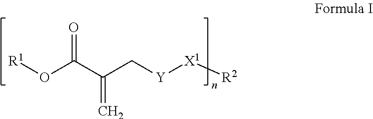
in which the variables have the following meanings:
R¹ is hydrogen, an aliphatic linear or branched C₁-C₁₅ alkyl radical which can be interrupted by one or more O or S, and/or can be unsubstituted or substituted by one or more C₁-C₅ alkyl groups and/or OH, or is benzyl or phenyl which can be unsubstituted or substituted by one or more C₁-C₃ alkyl groups;
Y is S or SO₂;
X¹ is absent or is an aliphatic linear or branched C₁-C₁₅ alkyl radical which can be interrupted by an ester group (—CO—O—), urethane group (—O—CO—NH—), one or more O or S, and/or can be unsubstituted or substituted by one or more C₁-C₅ alkyl groups and/or OH;
R² is an aliphatic linear or branched C₁-C₂₀ alkyl radical which can be interrupted by an ester group (—CO—O—), urethane group (—O—CO—NH—), one or more O or S, and/or can be unsubstituted or substituted by 1 to 3 OH groups, or is a cycloaliphatic C₅-C₂₀ radical, an aromatic C₆-C₂₀ radical or an isocyanuric acid radical, each of which may be unsubstituted or substituted by one or more C₁-C₅ alkyl groups;
n is 1, 2 or 3.

2. The radically polymerizable composition according to claim 1, wherein the variables of formula I have the following meanings:
R¹ is an aliphatic linear or branched C₁-C₁₀ alkyl radical which can be interrupted by 1 to 3 O or S, and/or can be unsubstituted or substituted by 1 to 3 methyl groups;
Y is S or SO₂;
X¹ is absent or is an aliphatic linear or branched C₁-C₁₀ alkyl radical which can be interrupted by an ester group (—CO—O—), urethane group (—O—CO—NH—), 1 to 3 O or S, and/or can be unsubstituted or substituted by 1 to 3 methyl groups;
R² is an aliphatic linear or branched C₁-C₁₆ alkyl radical which can be interrupted by an ester group (—CO—O—), urethane group (—O—CO—NH—), 1 to 3 O or S, and/or can be substituted by an OH group, or is an aromatic C₆-C₂₀ radical which is unsubstituted or substituted by a methyl group;
n is 1 or 2.

3. The radically polymerizable composition according to claim 2, wherein the variables of formula I have the following meanings:
R¹ is an aliphatic linear or branched C₁-C₆ alkyl radical which can be interrupted by one or more O or S;
Y is S or SO₂;
X¹ not applicable;
R² is an aliphatic linear or branched C₁-C₁₂ alkyl radical which can be interrupted by an ester group (—CO—O—), urethane group (—O—CONH—), 1 to 2 O or S, and/or can be unsubstituted or substituted by an OH group, or is an aromatic C₆-C₂₀ radical;
n is 1.

4. The radically polymerizable composition according to claim 1, wherein the urethane group-containing (meth)acrylate monomer is a monomer or mixture of monomers having a urethane group content of 0.01 to 5 mmol/g.

5. The radically polymerizable composition according to claim 4, wherein the urethane group-containing (meth)acrylate monomer is a urethane di(meth)-acrylate.

6. The radically polymerizable composition according to claim 1, comprising as fracture toughness modifier a block copolymer and/or polymer particles having a core-shell structure.

7. The radically polymerizable composition according to claim 6, comprising as block copolymer an AB diblock or an ABA triblock copolymer, wherein
the A block is an oligomer composed of one or more of the following monomers: cyclic aliphatic esters or ethers, arylene oxide, alkylene oxide, radically polymerizable monomers, and
the B block is a polysiloxane and/or a polyvinyl and/or a polyalkene and/or a polydiene oligomer and/or a hydrogenated polydiene oligomer.

8. The radically polymerizable composition according to claim 7, wherein
the A block is a polymerizate of caprolactone, 2,6-dialkyl-1,4-phenylene oxide, and
the B block is a polydiene oligomer, a hydrogenated polydiene oligomer, polyvinylalkanoate oligomer or a polysiloxane oligomer according to the formula-O—(SiR¹²₂—O)ₚ— in which
R¹² is a linear C₁-C₂₀ alkyl, branched C₃-C₁₂ alkyl or C₆-C₂₀ aryl group, wherein the individual R¹² radicals may be the same or different, and
p is a number from 3 to 100.

9. The radically polymerizable composition according to claim 1, which comprises
(a) 1 to 30 wt. % of at least one transfer reagent of formula I,
(b) 1 to 97.9 wt. % of the at least one urethane group-containing (meth)acrylate monomer,
(c) 0 to 20 wt. % of one or more mono(meth)acrylates,
(d) 0 to 50 wt. % of one or more di(meth)acrylates without urethane groups,
(e) 1 to 10 wt. % of the at least one fracture toughness modifier,
(f) optionally 10 to 5000 ppm of one or more inhibitors,
(g) 0.1 to 5.0 wt. % of at least one photoinitiator for the radical polymerization,
(h) 0 to 30 wt. % of one or more inorganic or organic fillers,
(i) 0 to 30 wt. % of one or more additives,
where all percentages refer to the total mass of the composition.

10. The radically polymerizable composition according to claim 9, which comprises
(a) 2 to 25 wt. % of the at least one transfer reagent of formula I,
(b) 20 to 95.84 wt. % of at least one urethane di(meth)acrylate,
(c) 0 to 10 wt. % of the one or more mono(meth)acrylates,
(d) 0 to 40 wt. % of the one or more di(meth)acrylates without urethane groups,
(e) 2 to 8 wt. % of the at least one fracture toughness modifier,
(f) 100 to 4000 ppm of at least one phenolic inhibitor,
(g) 0.15 to 4.0 wt. % of the at least one photoinitiator for the radical polymerization,
(h) 0 to 20 wt. % of the one or more inorganic or organic fillers,
(i) 0 to 25 wt. % of the one or more additives,
in each case based on the total mass of the composition.

11. The radically polymerizable composition according to claim 1, which does not contain monofunctional monomers and/or polymer particles having a core-shell structure.

12. A process for the manufacture of dental moldings, comprising the following steps:
(i) creating a virtual image of a tooth situation by directly or indirectly digitizing the tooth or teeth to be restored on the computer,
(ii) constructing a model of a dental restoration or prosthesis on the computer using the virtual image,
(iii) building up the dental restoration or prosthesis layer by layer by polymerization of a composition according to claim 1 by selective light irradiation.
